(19) United States
(12) Patent Application Publication
(10) Pub. No.: US 2005/0000790 A1
(43) Pub. Date: Jan. 6, 2005

(54) METHOD FOR ISOLATING AMINES FROM AQUEOUS SOLUTIONS

(76) Inventors: Karl Beck, Ostringen (DE); Torsten Freund, Limburgerhof (DE); Bernd Gerber, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/494,042

(22) PCT Filed: Nov. 12, 2002
(86) PCT No.: PCT/EP02/12603

(30) Foreign Application Priority Data
Nov. 18, 2001 (DE) 10156131.8

(51) Int. Cl. B01D 3/42; C07C 51/44

(52) U.S. Cl. 203/2; 203/14; 203/99; 203/DIG. 19; 203/75; 203/77; 203/78; 203/80

(57) ABSTRACT

A process for the isolation of amines from aqueous solutions by continuous distillation of aqueous solutions of the amines by a two-pressure method of rectifying aqueous solutions of amines which form with water an azeotrop, the composition of which is pressure-dependent, in two columns at different pressures provided that: a) the pressure in the second column is set to a value at least 0.5 bar higher than in the first column, b) the aqueous amine solution is fed, as a side stream, to the first column, and water and portions which boil at a higher temperature than the amine or which are non-volatile are removed at the base of the column, c) a mixture of amine and water is withdrawn at the head of the first column and fed, as a side stream, to the second column, d) an azeotrope of water and amine is withdrawn at the head of the second column and recycled to the first column, and (e) the purified amine is discharged at the base of the second column.

The invention relates to a process for the isolation of amines from aqueous solutions by continuous distillation of aqueous solutions of the amines by a two-pressure method of rectifying homogeneous azeotropic binary mixtures.

Two liquids which are completely soluble in each other and form azeotropes whose composition is pressure-dependent, can be separated by distillation without the assistance of an additive. Such a separation process involving rectification of liquid blends in two columns operated at different pressures are referred to as two-pressure processes, cf Klaus Sattler, “Thermische Trennverfahren, Grundlagen, Auslegung, Apparate”, 2nd Edition, Verlag Chemie, Weinheim, New York, Basel, Cambridge, Tokyo, 1995, pp. 147 et seq.

In practice there are obtained aqueous solutions of amines from which the amines have to be isolated in anhydrous form. Since the amines form an azeotrope with water, they cannot be readily isolated from aqueous solutions in anhydrous form by distillation. The recovery of pure amines from aqueous, salty solutions is an important factor when measuring the economical value of processes in which the amines are used as bases. In this process the amines must be recovered from the aqueous solutions of their ammonium salts formed during the reaction.

It is an object of the present invention to provide a process for the isolation of amines in substantially anhydrous form from aqueous solutions of said amines.

This object is achieved in a process for the isolation of amines from aqueous solutions by continuous distillation of aqueous solutions of the amines by a two-pressure method of rectifying homogeneous azeotropic binary mixtures, when aqueous solutions of amines forming an azeotrop with water, the composition of which is pressure-dependent, are distilled in two columns at different pressures, wherein
a) the pressure in the second column is set to a value at least 0.5 bar higher than in the first column,
b) the aqueous amine solution is fed as a side stream to the first column, and water and portions which boil at a temperature higher than the boiling point of the amines or which are non-volatile are removed at the base of the column,
c) a mixture of amine and water is withdrawn at the head of the first column and is passed as a side stream to the second column,
d) an azeotrope of water and amine is withdrawn at the head of the second column and is recycled to the first column, and
e) the purified amines are discharged at the base of the second column.

In a preferred embodiment of the process of the invention, the energy required to vaporize the azeotrope in the first column is introduced by passing steam into the lower part of this column and removing water and portions which boil at a higher temperature than the amines or which are non-volatile from the base of the column and removing highly volatile portions boiling at a lower temperature than the amines from the upper part of the first column. The pressure in the first column is set, for example, to values of 10 mbar to 10 bar and is usually from 0.1 to 3 bar. Particular preference is given to a mode of operation involving the use of atmospheric pressure in the first column.

In the preferred embodiment of the invention, the pressure in the second column is set to a value at least 1 bar higher than in the first column. In the particularly preferred embodiment of the invention the pressure in the second column is at least 6 bar higher than in the first column. The pressures and other conditions used for distillation (eg, temperature of the heat transfer medium such as cooling water, brine, or steam, the diameter of the columns and the size of the return stream between the columns) are preferably such that with regard to process engineering and economical aspects an optimal solution for the isolation of the amines is achieved.

According to the present invention, all amines which form with water an azeotrop whose composition is pressure-dependent, can be isolated in the process of the invention in virtually anhydrous form. The water content of the amines isolated from the aqueous solutions is, for example, from 10 to 200 ppm, and preferably from 20 to 60 ppm. In the process of the invention, the amines used are preferably N,N-dimethylisopropylamine, methyl-n-butylamine, or n-butylamine. Particular preference is given to the use of N,N-dimethylisopropylamine.

Such amines are used, for example, in substantially anhydrous form in the aforementioned process for the production of, say, alkyl ketene dimers. The resultant ammonium salts are separated and treated with aqueous solutions of bases in order to liberate the amines from the corresponding ammonium salts. Suitable bases are, for example, sodium hydroxide solution, potash lye, calcium hydroxide, and barium hydroxide.

Thus, for example, aqueous sodium hydroxide solution is added to an aqueous solution of N,N-dimethylisopropylammonium hydrochloride in a concentration such as to give a pH of the aqueous ammonium salt solution of at least 11. By this means N,N-dimethylisopropylamine is liberated from the ammonium salt solution and sodium chloride formed. This mixture can then be continuously distilled by the method of the invention to isolate N,N-dimethylisopropylamine in virtually anhydrous form.

Aqueous solutions of the amines, which may optionally still contain impurities, such as low-boiling components or neutral salts such as sodium chloride, can be distilled, for example, by the two-pressure method as follows: an aqueous, neutral salt-containing solution of amines which form with water an azeotrope whose composition is pressure-dependent is continuously fed as a side stream to a first column operated under atmospheric pressure. The side stream can, for example, be fed to the center of the column. The column has separating devices comprising plates, preferably sieve trays. The number of theoretical plates of the column is, eg, from 5 to 15, preferably from 8 to 12. The aqueous amine solution to be distilled and fed to the first column, can have a temperature ranging from 20° to 80°, preferably from 45° to 60° C. have. The energy necessary for distillation can be applied to the system with the aid of an evaporator at the base of the column or alternatively by the introduction of steam. Direct introduction of steam into the column, eg, at the lower end the column, is preferred. This avoids the formation of caked material in the column. The bottoms of the first column contain water and portions which boil at a higher temperature than the amines or are non-volatile, such as neutral salts. The overheads from the first column, comprising a mixture of amine and water (eg, 95 wt % of amine and 5 wt % of water), are continuously withdrawn and passed as a side stream to, eg, the upper half of the second column, in which the pressure is 7 bar, for example. An almost anhydrous amine is isolated at the bottom of the second column, likewise continuously. At the head of the second column there is continuously withdrawn an azeotrope of water and amine (eg, 89 wt % of amine and 11 wt % of water) which is recycled, for example, to the upper half of the first column. Highly volatile portions may optionally be continuously removed from the upper part of the first column. The virtually anhydrous amine can be reused, for example, in the synthesis of alkyl ketene dimers from carboxylic chlorides.

The reflux ratio is, for example, in the first column from 0.5 to 3, preferably from 0.5 to 2 and in the second column from 1 to 4, preferably from 1 to 2.

### EXAMPLE

The dimethylisopropylammmonium chloride formed during the production of alkyl ketene dimer from a long-chain carboxylic chloride (eg, stearoyl chloride) and N,N-dimethylisopropylamine in the form of a ca 50 wt % strength aqueous solution is set to pH 11 by the addition of 25 wt % strength aqueous sodium hydroxide solution and is heated to a temperature of 50° C. and continuously fed, as a side stream, to a column having 25 sieve trays and operated under atmospheric pressure. Heating is effected by continuous introduction of live steam having a temperature of ca 150° C. into the lower region of the column. The steam rate is ca 20% of the rate at which the amine solution is fed to the first column. From the base of the first column there are removed water, neutral salts such as sodium chloride, excess sodium hydroxide solution, and ca 10 ppm of N,N-dimethylisopropylamine. At the head the column there is formed a mixture of almost azeotropic composition (95 wt % of N,N-dimethylisopropylamine and 5 wt % of water). The condensed vapors are passed, as approximately equal partial streams, back to the first column and continuously, as a feed side stream, to the upper half of the second column, which is operated under a pressure of 7 bar absolute and has 30 sieve trays.

In the enriching zone of the first column there is a side flue, through which impurities are continuously withdrawn which are more volatile than water.

The bottom of the second column is heated by means of an evaporator. At the bottom of the second column, pure N,N-dimethylisopropylamine containing ca 40 ppm of water is continuously withdrawn. It can be directly reused, eg, for the synthesis of alkyl ketene dimers of carboxylic chlorides.

At the head of the second column there is obtained an azeotrope containing 89 wt % of N,N-dimethylisopropylamine and 11 wt % of water. The reflux ratio in the second column is 1.5. The azeotrope formed at the head of the second column is fed to the first column continuously as a side stream above the point at which the aqueous N,N-dimethylisopropylamine solution is metered. The condenser of the second column is degassed into the enriching section of the first column, which is operated under standard pressure.

1. A process for the isolation of an amine from an aqueous solution by continuous distillation of an aqueous solution of the amine by a two-pressure method of rectifying homogeneous azeotropic binary mixtures, wherein an aqueous solution of an amine which forms with water an azeotrop, the composition of which is pressure-dependent, is distilled in two columns at different pressures provided that:
a) the pressure in the second column is set to a value at least 0.5 bar higher than in the first column,
b) the aqueous amine solution is fed, as a side stream, to the first column, and water and portions which boil at a higher temperature than the amine or which are non-volatile are removed at the base of the column,
c) a mixture of amine and water is withdrawn at the head of the first column and fed, as a side stream, to the second column,
d) an azeotrope of water and amine is withdrawn at the head of the second column and recycled to the first column, and
(e) the purified amine is discharged at the base of the second column.

2. A process as defined in claim 1, wherein, in the first column, the energy necessary for vaporization of the azeotrope is introduced by feeding in steam to the lower part of this column, and water and portions which boil at a higher temperature than the amine or which are non-volatile are removed at the bottom of the column, and highly volatile portions which boil at a lower temperature than the amine are removed from the upper part of the first column.

3. A process as defined in claim 1, wherein the pressure in the first column is set to a value of from 10 mbar to 10 bar.

4. A process as defined in claim 1, wherein the pressure in the first column is set to atmospheric pressure.

5. A process as defined in claim 1, wherein the pressure in the second column is set to a value at least 6 bar higher than in the first column.

6. A process as defined in claim 1, wherein the pressure in the second column is set to a value at least 1 bar higher than in the first column.

7. A process as defined in claim 1, wherein the amine used is N,N-dimethylisopropylamine, methyl-n-butylamine or n-butylamine.

8. A process as defined in claim 1, wherein, for said continuous distillation, an aqueous solution of an amine is used which is obtained by liberating the amine from an aqueous amine salt solution with a base.

9. A process as defined in claim 1, wherein the amine used is N,N-dimethylisopropylamine.

10. A process as defined in claim 1, wherein the separating devices used are plates.

11. A process as defined in claim 1, wherein the separating devices used are sieve trays.
